(19) United States
(12) Patent Application Publication
(10) Pub. No.: US 2017/0264994 A1
(43) Pub. Date: Sep. 14, 2017

(54) Audio Sleep Mask Apparatus

(71) Applicants: Cynthia Callendar Gordon, Las Vegas, NV (US); Marc Charles Gordon, Las Vegas, NV (US)

(72) Inventors: Cynthia Callendar Gordon, Las Vegas, NV (US); Marc Charles Gordon, Las Vegas, NV (US)

(21) Appl. No.: 15/067,133

(22) Filed: Mar. 10, 2016

(51) Int. Cl. H04R 1/10; A61M 21/02; H04R 1/02; A61F 9/04

(52) U.S. Cl. CPC H04R 1/1091; A61F 9/04; A61M 21/02; H04R 1/026; A61M 2021/0027

(57) ABSTRACT

An audio sleep mask apparatus provides, in at least one embodiment, a sleep mask removably engagable about a head of a user, the sleep mask comprising a first panel joined with an opposing second panel. The sleep mask provides a face portion positionable over a pair of eyes of the user, and opposing left and right side portions positionable over respective ears of the user. The sleep mask defines an at least one compartment between the first and second panels sized and positioned for removably receiving a wireless speaker system therewithin, the wireless speaker system comprising a left speaker, a right speaker and a wireless receiver. With the wireless speaker system positioned within the at least one compartment, the sleep mask is capable of being comfortably worn such that the left speaker is positioned proximal a left ear and the right speaker is positioned proximal a right ear.

### BACKGROUND

The subject of this patent application relates generally to sleep aids, and more particularly to an audio sleep mask apparatus.

Applicant(s) hereby incorporate herein by reference any and all patents and published patent applications cited or referred to in this application.

By way of background, the use of various types of audio—i.e., music, white noise, etc.—has proven to be very effective for sleep aid purposes. However, traditional personal audio delivery devices, such as headphones and earbuds, typically have rigid plastic or metal components that tend to create discomfort if worn while lying down or sleeping. Thus, there is a need for a personal audio delivery device that can be worn comfortably while sleeping.

Aspects of the present invention fulfill these needs and provide further related advantages as described in the following summary.

### SUMMARY

Aspects of the present invention teach certain benefits in construction and use which give rise to the exemplary advantages described below.

The present invention solves the problems described above by providing an audio sleep mask apparatus. In at least one embodiment, the apparatus provides a sleep mask removably engagable about, and capable of substantially conforming to, a head of a user, the sleep mask comprising a first panel joined with an opposing second panel. The sleep mask provides a face portion positionable over a pair of eyes of the user, and opposing left and right side portions positionable over respective ears of the user. Additionally, the sleep mask defines an at least one compartment between the first and second panels sized and positioned for removably receiving a wireless speaker system therewithin, the wireless speaker system comprising a left speaker, a right speaker and a wireless receiver. Thus, with the wireless speaker system positioned within the at least one compartment, the sleep mask is capable of being comfortably worn by the user such that the left speaker is positioned proximal a left ear and the right speaker is positioned proximal a right ear.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a partially exploded perspective view of an exemplary wireless audio sleep mask apparatus, in accordance with at least one embodiment;

FIG. 2 is a front view thereof, in accordance with at least one embodiment;

FIG. 3 is a top view thereof, in accordance with at least one embodiment;

FIG. 4 is a cross-sectional view thereof, taken along line 4-4 of FIG. 3; and

FIG. 5 is a perspective view of the exemplary sleep mask apparatus as worn by a user.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

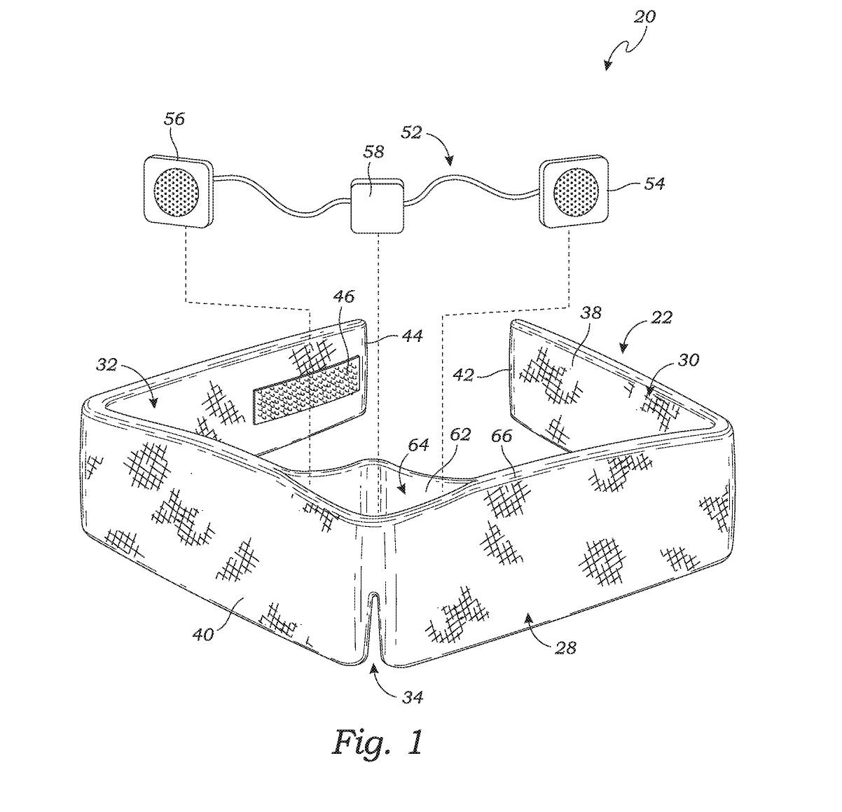

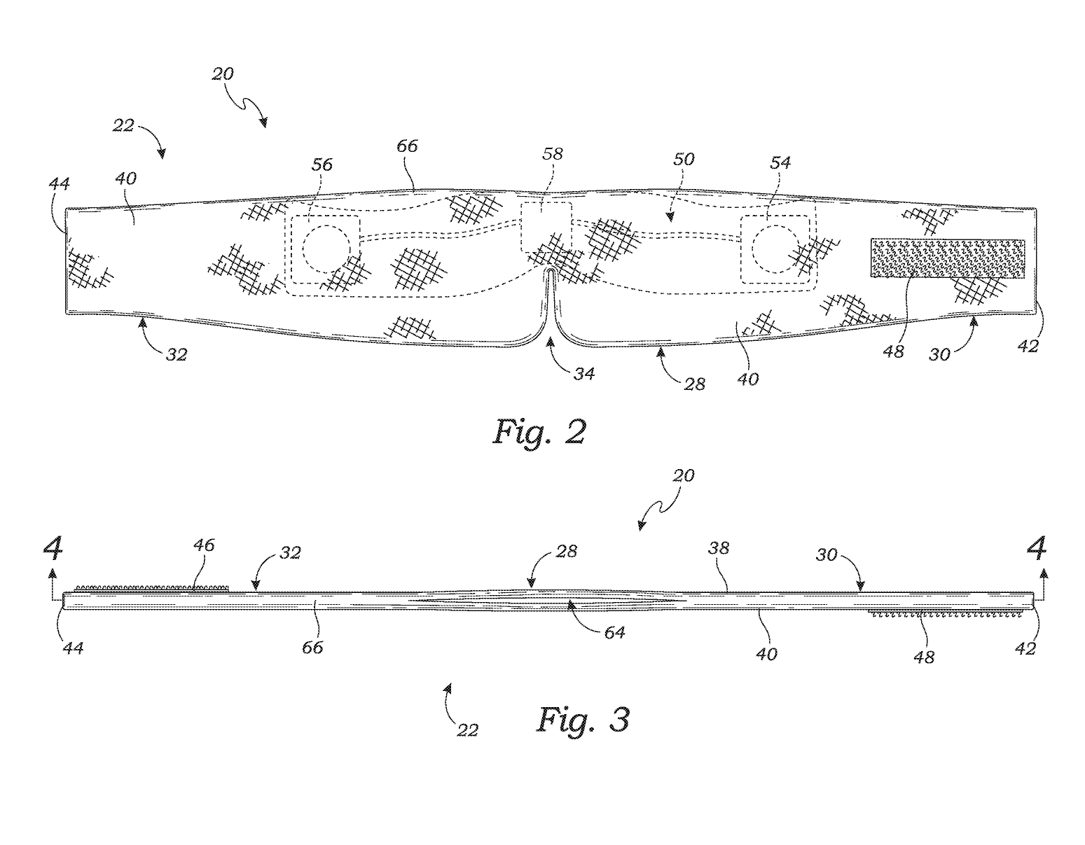

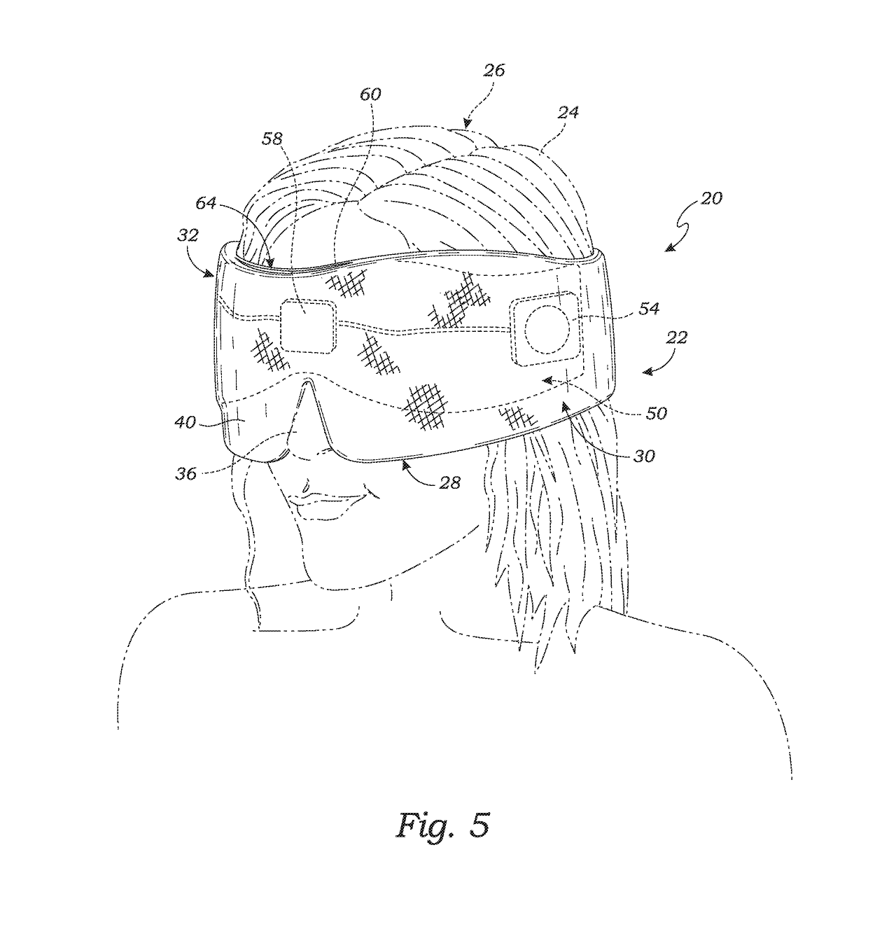

Turning now to FIG. 1, there is shown a perspective view of an exemplary embodiment of an audio sleep mask apparatus 20. The apparatus 20 comprises, in the exemplary embodiment, a sleep mask 22 removably engagable about, and capable of substantially conforming to, a head 24 of a user 26 (FIG. 5). In at least one embodiment, as illustrated in FIGS. 1 and 2, the sleep mask 22 provides a face portion 28 positionable over a pair of eyes (not shown) of the user 26, and opposing left and right side portions 30 and 32 positionable over respective ears (not shown) of the user 26 when the sleep mask 22 is being worn. In at least one embodiment, the left and right side portions 30 and 32 are sized for substantially covering the user's ears. In a still further embodiment, the left and right side portions 30 and 32 are sized for completely covering the user's ears. Additionally, in at least one embodiment, the face portion 28 defines a nose cutout 34 for allowing a nose 36 of the user 26 to be substantially unobstructed when the sleep mask 22 is being worn.

With continued reference to FIG. 1, in at least one embodiment, the sleep mask comprises a first panel 38 and an opposing second panel 40 joined to the first panel 38. In the exemplary embodiment, the first and second panels 38 and 40 are joined using stitching; however, in further embodiments, any other mechanisms for permanently joining the first and second panels 38 and 40, now known or later developed—such as welding, adhesives, etc.—may be substituted. In still further embodiments, the first and second panels 38 and 40 may be removably engagable with one another using hook-and-loop fasteners, buttons, snaps, zippers or any other non-permanent engagement mechanisms now know or later developed—the purpose for which is discussed further below.

As mentioned above, the sleep mask 22 is removably engagable about the user's head 24. In at least one embodiment, a left free end 42 of the left side portion 30 is removably engagable with a right free end 44 of the right side portion 32 when the left and right free ends 42 and 44 selectively overlap one another. In at least one such embodiment, the first panel 38 provides a first engagement mechanism 46 proximal the left free end 42 while the second panel 40 provides a corresponding second engagement mechanism 48 (capable of temporarily mating with or otherwise engaging the first engagement mechanism 46) proximal the right free end 44. In at least one alternate embodiment, the first panel 38 provides the first engagement mechanism 46 proximal the right free end 44 while the second panel 40 provides the second engagement mechanism 48 proximal the left free end 42. In at least one embodiment, the first and second engagement mechanisms 46 and 48 are corresponding hook-and-loop fasteners; however, in further embodiments, any other non-permanent engagement mechanisms, now known or later developed, may be substituted. In at least one alternate embodiment (not shown), the left side portion 30 is integral with the right side portion 32 such that the left side portion 30, face portion 28 and right side portion 32 form a continuous loop. In such an alternate embodiment, the user 26 is capable of temporarily stretching and pulling the sleep mask 22 down in position around their head 24.

In at least one embodiment, one or both of the first and second panels 38 and 40 are constructed of a soft, relatively flexible material, such as a padded fabric. However, other materials or combinations of materials, now known or later developed, may be substituted in further embodiments that allow the first and second panels 38 and 40 to substantially carry out the functionality described herein. In at least one further embodiment, since only one of the first and second panels 38 and 40 contacts the user's head 24 when the sleep mask 22 is being worn, the other of the first and second panels 38 and 40 (i.e. an outer facing one of the first and second panels 38 and 40) may be constructed of a relatively more visually appealing material or combination of materials.

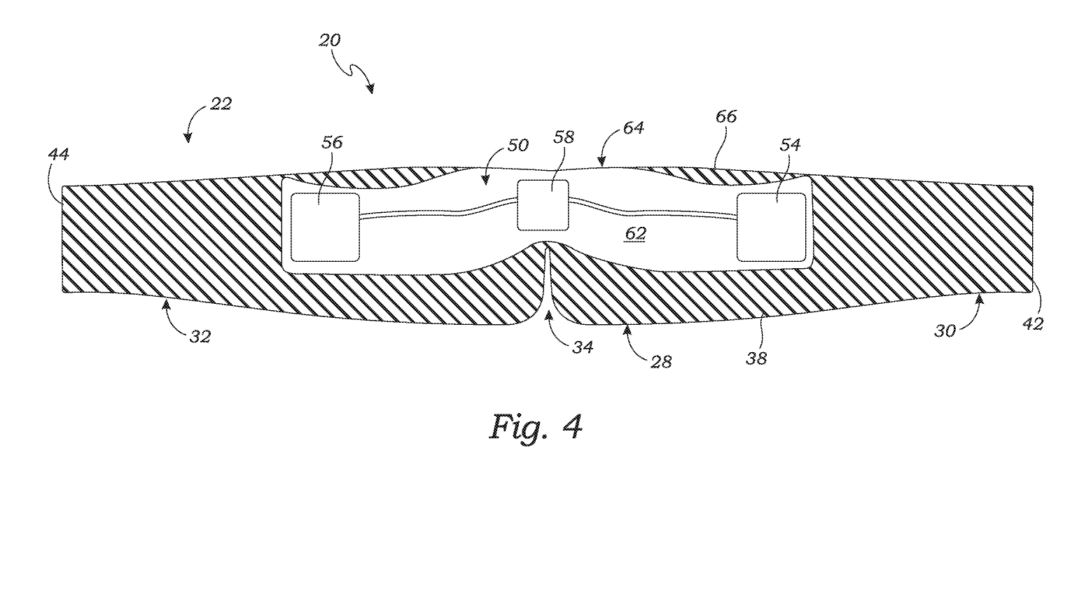

In at least one embodiment, as best shown in FIGS. 1 and 4, the sleep mask 22 defines an at least one compartment 50 between the first and second panels 38 and 40 sized and positioned for removably receiving a wireless speaker system 52 therewithin. In at least one such embodiment, the wireless speaker system 52 comprises a left speaker 54 and a right speaker 56 in wired communication with a wireless receiver 58. The wireless receiver 58 is in wireless communication with an at least one audio source (not shown), such as a stereo system, computer, digital music player, CD player, cassette player, portable music player, or any other device, now known or later developed, capable of wirelessly transmitting audio signals. Additionally, any wireless communication protocol or combination of protocols, now known or later developed, may be utilized by the wireless receiver 58, such as Bluetooth for example. In at least one alternate embodiment (not shown), one or both of the left and right speakers 54 and 56 may incorporate a wireless receiver therewithin, thereby eliminating the need for the separate wireless receiver 58 shown in the drawings.

In at least one embodiment, as illustrated best in FIGS. 4 and 5, the at least one compartment 50 is sized and configured for allowing the left speaker 54 to be positioned proximal the left ear of the user 26 and the right speaker 56 to be positioned proximal the right ear of the user 26, when the sleep mask 22 is being worn. Additionally, in at least one embodiment, the wireless receiver 58 is positioned proximal a forehead 60 of the user 26 so as to reduce the likelihood of the wireless receiver 58 causing discomfort to the user 26 while the user 26 is laying down. For similar reasons, in at least one embodiment, the left and right speakers 54 and 56 are relatively thin so as to better ensure the comfort of the user 26 while in any laying position. In at least one embodiment, an inner surface 62 of the at least one compartment 50 provides a relatively high friction material for assisting in retaining the components of the wireless speaker system 52 in their respective positions while within the compartment 50. In at least one alternate embodiment (not shown), the inner surface 62 of the at least one compartment 50 provides a left pocket positioned proximal the left ear of the user 26, and sized for removably receiving the left speaker 54, and a right pocket positioned proximal the right ear of the user 26, and sized for removably receiving the right speaker 56. In at least one further embodiment (not shown), the inner surface 62 of the at least one compartment 50 also provides a center pocket positioned proximal the forehead 60 of the user 26, and sized for removably receiving the wireless receiver 58. In still further embodiments, any other structure or mechanism (or combinations thereof) capable of assisting in retaining the components of the wireless speaker system 52 in their respective positions while within the at least one compartment 50, now known or later developed, may be utilized.

With continued reference to FIGS. 1 and 4, in at least one embodiment, the first and second panels 38 and 40 define an at least one opening 64 for allowing access into the at least one compartment 50. In at least one such embodiment, the at least one opening 64 is positioned along a top edge 66 of the sleep mask 22. It should be noted, in further embodiments, the at least one opening 64 may be positioned anywhere else on the sleep mask 22—whether along an edge of the sleep mask 22 or elsewhere on the first or second panels 38 or 40. In at least one such embodiment, the at least one opening 64 provides a closure mechanism (not shown) for selectively closing the opening 64 and preventing the wireless speaker system 52 from unintentionally falling out of the corresponding compartment 50. In at least one embodiment, the closure mechanism is a hook-and-loop fastener; however, in further embodiments, any other non-permanent engagement mechanisms, now known or later developed, may be substituted. In at least one alternate embodiment, rather than provide at least one opening 64, the first and second panels 38 and 40 are instead removably engagable with one another, as mentioned above; thereby allowing access into the at least one compartment 50 when the first and second panels 38 and 40 are separated.

In at least one embodiment, the wireless speaker system 52 is able to be removed from the at least one compartment 50, in part, so that the wireless receiver 58 may be selectively recharged as needed. However, in at least one embodiment, the wireless receiver 58 may be recharged while it remains in the at least one compartment 50. In at least one such embodiment, the wireless receiver 58 may utilize inductive charging or another wireless charging technology, now known or later developed. In at least one other such embodiment (not shown), where the wireless receiver 58 relies on a wire-based charging technology, the sleep mask 22 may provide a charging aperture sized and positioned for allowing an appropriate charging wire to interface with the wireless receiver 58 while it is positioned within the at least one compartment 50. In such embodiments, where removal of the wireless receiver 58 is unnecessary, the at least one opening 64 may be omitted such that the wireless speaker system 52 is permanently retained within the at least one compartment 50.

Thus, with the wireless speaker system 52 positioned within the at least one compartment 50, the sleep mask 22 is capable of being comfortably worn by the user 26 (FIG. 5) such that the left speaker 54 is positioned proximal the left ear of the user 26 and the right speaker 56 is positioned proximal the right ear of the user 26.

Aspects of the present specification may also be described as follows:

1. An audio sleep mask apparatus comprising: a sleep mask removably engagable about, and capable of substantially conforming to, a head of a user, the sleep mask comprising a first panel joined with an opposing second panel; the sleep mask providing a face portion positionable over a pair of eyes of the user, and opposing left and right side portions positionable over respective ears of the user; and the sleep mask defining an at least one compartment between the first and second panels sized and positioned for removably receiving a wireless speaker system therewithin, the wireless speaker system comprising a left speaker, a right speaker and a wireless receiver; whereby, with the wireless speaker system positioned within the at least one compartment, the sleep mask is capable of being comfortably worn by the user such that the left speaker is positioned proximal a left ear and the right speaker is positioned proximal a right ear.

2. The audio sleep mask apparatus according to embodiment 1, wherein the face portion defines a nose cutout for allowing a nose of the user to be substantially unobstructed when the sleep mask is engaged about the head of the user.

3. The audio sleep mask apparatus according to embodiments 1-2, wherein a left free end of the left side portion is removably engagable with a right free end of the right side portion when said left and right free ends selectively overlap one another.

4. The audio sleep mask apparatus according to embodiments 1-3, wherein the left side portion is integral with the right side portion such that the left side portion, face portion and right side portion form a continuous loop.

5. The audio sleep mask apparatus according to embodiments 1-4, wherein the first and second panels are made of a relatively flexible fabric.

6. The audio sleep mask apparatus according to embodiments 1-5, wherein the first and second panels define an at least one opening for allowing access into the at least one compartment.

7. The audio sleep mask apparatus according to embodiments 1-6, wherein the at least one opening is positioned along a top edge of the sleep mask.

8. The audio sleep mask apparatus according to embodiments 1-7, wherein the at least one opening provides a closure mechanism configured for selectively closing said opening.

9. The audio sleep mask apparatus according to embodiments 1-8, wherein an inner surface of the at least one compartment provides a relatively high friction material for assisting in retaining the wireless speaker system in position while within said compartment.

10. The audio sleep mask apparatus according to embodiments 1-9, wherein an inner surface of the at least one compartment provides: a left pocket positioned proximal the left ear of the user and sized for removably receiving the left speaker; and a right pocket positioned proximal the right ear of the user and sized for removably receiving the right speaker.

11. The audio sleep mask apparatus according to embodiments 1-10, wherein the inner surface of the at least one compartment further provides a center pocket positioned proximal a forehead of the user and sized for removably receiving the wireless receiver.

12. The audio sleep mask apparatus according to embodiments 1-11, wherein each of the left and right speakers are in wired communication with the wireless receiver.

13. An audio sleep mask apparatus comprising: a sleep mask removably engagable about a head of a user, the sleep mask comprising a first panel joined with an opposing second panel; the sleep mask providing a face portion positionable over a pair of eyes of the user, and opposing left and right side portions positionable over respective ears of the user; the sleep mask defining an at least one compartment between the first and second panels; and a wireless speaker system removably positionable within the at least one compartment, the wireless speaker system comprising a left speaker, a right speaker and a wireless receiver; whereby, with the wireless speaker system positioned within the at least one compartment, the sleep mask is capable of being comfortably worn by the user such that the left speaker is positioned proximal a left ear and the right speaker is positioned proximal a right ear.

14. An audio sleep mask apparatus comprising: a sleep mask removably engagable about a head of a user, the sleep mask comprising a first panel joined with an opposing second panel; the sleep mask providing a face portion positionable over a pair of eyes of the user, and opposing left and right side portions positionable over respective ears of the user; the face portion defining a nose cutout for allowing a nose of the user to be substantially unobstructed when the sleep mask is engaged about the head of the user; the sleep mask defining an at least one compartment between the first and second panels; the first and second panels defining an at least one opening for allowing access into the at least one compartment; and a wireless speaker system removably positionable within the at least one compartment, the wireless speaker system comprising a left speaker, a right speaker and a wireless receiver; whereby, with the wireless speaker system positioned within the at least one compartment, the sleep mask is capable of being comfortably worn by the user such that the left speaker is positioned proximal a left ear and the right speaker is positioned proximal a right ear.

In closing, regarding the exemplary embodiments of the present invention as shown and described herein, it will be appreciated that a wireless audio sleep mask apparatus is disclosed. Because the principles of the invention may be practiced in a number of configurations beyond those shown and described, it is to be understood that the invention is not in any way limited by the exemplary embodiments, but is generally directed to a wireless audio sleep mask apparatus and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention. Furthermore, the various features of each of the above-described embodiments may be combined in any logical manner and are intended to be included within the scope of the present invention.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term.

Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

It should be understood that the logic code, programs, modules, processes, methods, and the order in which the respective elements of each method are performed are purely exemplary. Depending on the implementation, they may be performed in any order or in parallel, unless indicated otherwise in the present disclosure.

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. An audio sleep mask apparatus comprising:
a sleep mask removably engagable about, and capable of substantially conforming to, a head of a user, the sleep mask comprising a first panel joined with an opposing second panel;
the sleep mask providing a face portion positionable over a pair of eyes of the user, and opposing left and right side portions positionable over respective ears of the user; and
the sleep mask defining an at least one compartment between the first and second panels sized and positioned for removably receiving a wireless speaker system therewithin, the wireless speaker system comprising a left speaker, a right speaker and a wireless receiver;
whereby, with the wireless speaker system positioned within the at least one compartment, the sleep mask is capable of being comfortably worn by the user such that the left speaker is positioned proximal a left ear and the right speaker is positioned proximal a right ear.

2. The audio sleep mask apparatus of claim 1, wherein the face portion defines a nose cutout for allowing a nose of the user to be substantially unobstructed when the sleep mask is engaged about the head of the user.

3. The audio sleep mask apparatus of claim 1, wherein a left free end of the left side portion is removably engagable with a right free end of the right side portion when said left and right free ends selectively overlap one another.

4. The audio sleep mask apparatus of claim 1, wherein the left side portion is integral with the right side portion such that the left side portion, face portion and right side portion form a continuous loop.

5. The audio sleep mask apparatus of claim 1, wherein the first and second panels are made of a relatively flexible fabric.

6. The audio sleep mask apparatus of claim 1, wherein the first and second panels define an at least one opening for allowing access into the at least one compartment.

7. The audio sleep mask apparatus of claim 6, wherein the at least one opening is positioned along a top edge of the sleep mask.

8. The audio sleep mask apparatus of claim 6, wherein the at least one opening provides a closure mechanism configured for selectively closing said opening.

9. The audio sleep mask apparatus of claim 1, wherein an inner surface of the at least one compartment provides a relatively high friction material for assisting in retaining the wireless speaker system in position while within said compartment.

10. The audio sleep mask apparatus of claim 1, wherein an inner surface of the at least one compartment provides:
a left pocket positioned proximal the left ear of the user and sized for removably receiving the left speaker; and
a right pocket positioned proximal the right ear of the user and sized for removably receiving the right speaker.

11. The audio sleep mask apparatus of claim 10, wherein the inner surface of the at least one compartment further provides a center pocket positioned proximal a forehead of the user and sized for removably receiving the wireless receiver.

12. The audio sleep mask apparatus of claim 1, wherein each of the left and right speakers are in wired communication with the wireless receiver.

13. An audio sleep mask apparatus comprising:
a sleep mask removably engagable about a head of a user, the sleep mask comprising a first panel joined with an opposing second panel;
the sleep mask providing a face portion positionable over a pair of eyes of the user, and opposing left and right side portions positionable over respective ears of the user;
the sleep mask defining an at least one compartment between the first and second panels; and
a wireless speaker system removably positionable within the at least one compartment, the wireless speaker system comprising a left speaker, a right speaker and a wireless receiver;
whereby, with the wireless speaker system positioned within the at least one compartment, the sleep mask is capable of being comfortably worn by the user such that the left speaker is positioned proximal a left ear and the right speaker is positioned proximal a right ear.

14. An audio sleep mask apparatus comprising:
a sleep mask removably engagable about a head of a user, the sleep mask comprising a first panel joined with an opposing second panel;
the sleep mask providing a face portion positionable over a pair of eyes of the user, and opposing left and right side portions positionable over respective ears of the user;
the face portion defining a nose cutout for allowing a nose of the user to be substantially unobstructed when the sleep mask is engaged about the head of the user;
the sleep mask defining an at least one compartment between the first and second panels;
the first and second panels defining an at least one opening for allowing access into the at least one compartment; and
a wireless speaker system removably positionable within the at least one compartment, the wireless speaker system comprising a left speaker, a right speaker and a wireless receiver;
whereby, with the wireless speaker system positioned within the at least one compartment, the sleep mask is capable of being comfortably worn by the user such that the left speaker is positioned proximal a left ear and the right speaker is positioned proximal a right ear.
